(19) United States
(12) Plant Patent Application Publication
(10) Pub. No.: US 2008/0235836 P1
(43) Pub. Date: Sep. 25, 2008

(54) Kalanchoe plant named 'Forever Midi Coral'

(75) Inventor: Lyndon W. Drewlow, Lompoc, CA (US)

Correspondence Address: THE WEBB LAW FIRM, P.C., 700 KOPPERS BUILDING, 436 SEVENTH AVENUE, PITTSBURGH, PA, 15219, US

(73) Assignee: The Paul Ecke Ranch, Encinitas, CA (US)

(21) Appl. No.: 11/728,029

(22) Filed: Mar. 23, 2007

(51) Int. Cl. A01H 5/00

(52) U.S. Cl. PLT/335

(57) ABSTRACT

‘Forever Midi Coral’ is a new variety of kalanchoe that exhibits coral-colored flowers and small leaves with a long keeping quality.

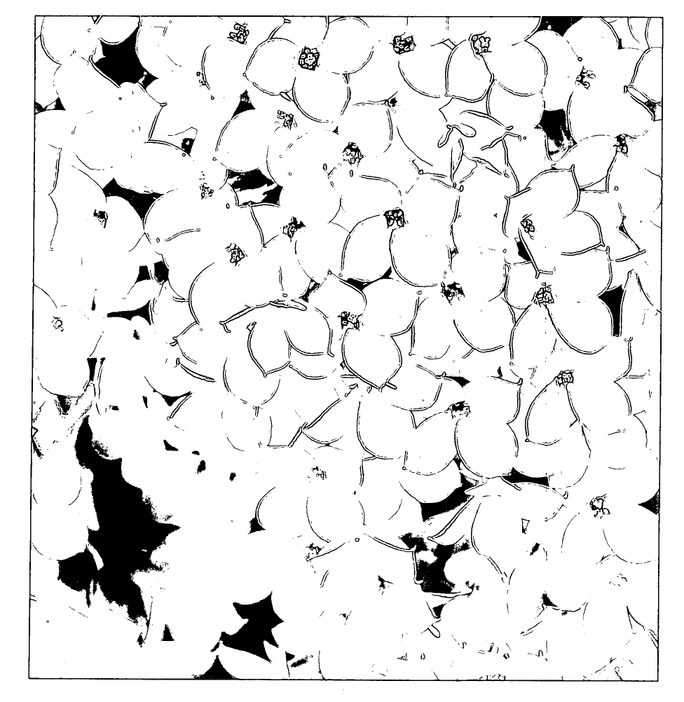

### BOTANICAL CLASSIFICATION

Kalanchoe blossfeldiana

### VARIETAL DENOMINATION ‘Forever Midi Coral’

### BACKGROUND OF THE INVENTION

The present invention comprises a new and distinct cultivar of Kalanchoe blossfeldiana known by the varietal name ‘Forever Midi Coral’. The new variety was discovered in Ashtabula, Ohio. The new variety is a result of cross breeding Seedling #95-362-10 (female parent) (unpatented) and Seedling #94-225-5 (male parent) (unpatented). The purpose of the breeding program was to develop a highly self-branching cultivar with a full, mounded flower cluster that was coral in color and had small leaves. Comparisons between the new variety and its parents cannot be made, as the parental varieties no longer exist. The new variety was first asexually reproduced by tip cuttings in Lompoc, Calif. The new variety has been trial and field tested and has been found to retain its distinctive characteristics and remain true to type through successive propagations. Characteristics that distinguish the new variety from others known to the breeder include:
Mounded and compact growing habit;
Coral flower color with a small white eye in the center;
Early to flower, 9 to 9½ weeks from the start under short days;
Able to be finished in 10 cm pots without growth regulators;
Small leaf size;
Does not heat delay in night temperatures above 22° C.;
Long keeping quality with indeterminant growth habit; and
Highly self-branched without pinching.

The new variety was grown in a 10 cm container in Lompoc, Calif. in a greenhouse under Dynaglass with 30% shade and 3500 to 4000 foot candles and 150 PPM nitrogen. The new variety was grown under natural, short days having temperatures of 20-22° C. and night temperatures of 16-18° C. The new variety initiates roots in 10 days at 21° C. The time to produce a rooted plant is 21 days at 21° C. Its rooting habit is dense and freely branching with fine and fibrous grey-white roots.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Lompoc, Calif. The color readings were taken indoors under 3500-4000 foot candles of light. The new variety was grown in a 10 cm container and was twelve weeks from the start of short days when described. Color references are primarily to the 1966 R.H.S. Colour Chart of The Royal Horticultural Society of London.
Plant:
Form: Mounded
Height from soil to top of flowers: 15 cm
Plant diameter: 22 to 25 cm
Vigor: Vigorous
Branching habit: Highly self-branching
Main stem:
   Color.—146B.
   Texture.—Glabrous.
   Length.—11 cm.
   Diameter.—12 mm.
   Internode length.—14 mm.
   Strength.—Strong.
Lateral branches:
   Color.—146B.
   Texture.—Glabrous.
   Length.—Basal: 12-13 cm Upper: 8-10 cm.
   Diameter.—3 mm.
   Internode length.—3-8 mm.
   Strength.—Strong.
   Quantity.—8-10.
Foliage:
   Arrangement.—Opposite.
   Number of leaves per stem.—2.
   Size of leaf.—Length: 8-9 cm Width: 5.5-6.5 cm.
   Shape of leaf (generally).—Ovate.
   Shape of apex.—Acute.
   Shape of base.—Acute.
   Texture.—Glabrous, coriaceous and succulent.
   Aspect.—45% angle to stem.
   Margin type.—Crenate, moderately lobed.
   Fragrance.—Shallow cut.
   Color.—Young leaves: Upper surface: 146A Lower surface: 146B Mature leaves: Upper surface: 147A Lower surface: 147B.
   Petiole.—Length: 8-10 mm Diameter: 6 mm Color: 146B Texture: Glabrous.
   Veins.—Venation pattern: Pinnate Color: Upper surface: 147A Lower surface: 147B.
Flower:
Natural flowering season: In January, in Lompoc, Calif. — Short days start around October 15^{th}.
Flower type and habit: Single, upright, cyme inflorescence
Number of flowers per plant: 250 or more
Time of day of flower opening: Morning, but takes 3 to 5 days to fully open
Fragrance: None
Longevity: Up to 2 weeks depending on environment
Flower height: 13 mm including corolla tube
Flower diameter: 15 mm
Bud:
   Stage of development when described.—Just before opening.
   Shape.—Oblong.
   Diameter.—3 mm.
   Length.—15 mm.
   Color.—39A near tip on mature bud; 144C calyx area.
Corolla:
   Diameter.—4 mm.
   Depth.—18 mm.
   Tube.—Length: 12 mm Diameter: 4 mm.
Petals:
   Arrangement.—Whorl of 4 fused at base.
   Quantity.—4.
   Length.—8 mm; none fused part.
   Width.—6 mm.
   Shape.—Ovate to rounded.
   Apex.—Cuspidate.
   Base.—Acute.
   Margin.—Entire.
   Texture.—Smooth; soft.
   Color.—Upper surface: 61D Lower surface: 62C.
Sepals:
   Quantity.—4.
   Shape.—Lanceolate.
   Length.—8 mm.
   Width.—2 mm.
   Color.—145B.
Peduncles:
   Length.—20 mm.
   Diameter.—3 mm.
   Color.—146B.
   Texture.—Glabrous.
   Orientation.—45° angle.
   Strength.—Strong.
Pedicels:
   Length.—3 mm.
   Width.—1 mm.
   Color.—146B.
Disease resistance: Observed
Weather tolerance: Not applicable — Mostly indoor flowering, pot crop
Lasting quality:
   On plant.—2 weeks or longer.
   Of cut flowers.—Not applicable.
Seed production and description: None observed
Fruit production and description: None observed
Reproductive organs:
Stamens:
   Number per flower.—8.
   Filament length.—8 mm approximately, 4 mm fuses to corolla.
   Anthers.—Shape: Flat elliptical Length: Less than 1 mm Color: Yellow — Green.
   Pollen.—Color: Yellow Amount: Abundant.
Pistils:
   Length.—10 mm.
   Styles.—Length: 3 mm Color: 144B.
   Stigma.—Shape: Flat Color: White when mature Ovary color: 144A.

I claim:

1. A new and distinct variety of kalanchoe plant named ‘Forever Midi Coral’ as is herein described and illustrated.
